(19) United States
(12) Patent Application Publication
(10) Pub. No.: US 2026/0360498 A1
(43) Pub. Date: Nov. 12, 2026

(54) PRESCRIPTION DRUG AVAILABILITY ESTIMATION SYSTEM

(71) Applicant: Medical Security Card Company, LLC, Tucson, AZ (US)

(72) Inventor: Jon Olaf JENSEN, New Canaan, CT (US)

(21) Appl. No.: 19/670,674

(22) Filed: May 7, 2026

Related U.S. Application Data
(60) Provisional application No. 63/801,498, filed on May 7, 2025.

(51) Int. Cl. G16H 20/10; G06Q 40/08

(52) U.S. Cl. CPC G16H 20/10; G06Q 40/08

(57) ABSTRACT

A drug availability estimate system is provided that includes a database comprising prescription rate data information and manufacture rate data information for a plurality of prescription drugs. The system includes an estimation system that analyzes the prescription rate data and the manufacture rate data for a first prescription drug and estimates an availability of the first prescription drug during a prescription period. The estimation system also identifies times during the prescription period when the availability of the first prescription drug is less than a first threshold. The system also includes an alert system configured to send an alert comprising a notice that the first prescription drug is estimated to be unavailable for refill at some point during the prescription period.

### RELATED APPLICATION

The present application claims priority to U.S. patent application no. 63/801,498 filed May 7, 2025, which is incorporated herein by reference in its entirety.

### BACKGROUND

### Field of the Invention

The present disclosure relates to improvements in pharmaceutical supply chain management systems, and more particularly relates to a system and method for estimating prescription drug availability based on analysis of prescription rate data and manufacture rate data.

### Related Art

Prescription drug use has been steadily increasing in accordance with an increase in the number of available drugs to treat an increasing number of diagnosed ailments. Additionally, alternative uses of drugs designed for a particular ailment further increases the use of prescription drugs. Moreover, the overall success of healthcare and pharmaceutical industries has increased the average lifespan and therefore also increased the number of aging adults that are taking prescription drugs.

As with any supply and demand system, sometimes the demand for prescription drugs exceeds the supply. For example, raw materials to manufacture the drugs may be limited, legal issues may prevent the manufacture or transportation of the drugs, manufacturers may erroneously predict a smaller demand for the drug, and many other circumstances may result in an imbalance between the supply and the demand of prescription drugs. When such imbalances occur, drugs may become out-of-stock or restricted by the manufacturer and are therefore not available to patients with a prescription.

Conventional pharmaceutical supply chain management systems face several technical challenges that limit their effectiveness in addressing these supply and demand imbalances. Current systems lack the ability to proactively analyze and compare prescription rate data with manufacture rate data to anticipate future drug availability issues. As a result, healthcare providers are often unaware of potential drug shortages until the shortage has already occurred, at which point patients may be unable to obtain their prescribed medications for refills during a prescription period.

Another disadvantage of conventional systems is their inability to provide timely notifications to healthcare providers at the point of prescribing. Without access to predictive availability information, physicians may prescribe drugs that will become unavailable before the prescription period ends, leading to disruptions in patient care and the need for reactive rather than proactive prescription modifications. Additionally, existing systems do not adequately integrate prescription rate information with manufacture rate information from various data sources, whether public or proprietary, to generate meaningful availability estimates for specific time periods.

Therefore, what is needed is a system and method that overcomes these significant problems found in the conventional systems as described above.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

The present disclosure addresses the aforementioned problems of conventional pharmaceutical supply chain management systems that lack proactive analysis capabilities and fail to provide timely notifications regarding drug availability. By implementing an estimation system that analyzes and compares prescription rate data with manufacture rate data for prescription drugs, the disclosed system enables proactive identification of potential drug shortages before they occur. The estimation system estimates the availability of a prescription drug at each of a plurality of dates during a prescription period, thereby allowing healthcare providers to anticipate availability issues rather than discovering them after a shortage has already materialized. Additionally, the disclosed alert system provides timely notifications to healthcare providers at the point of prescribing when a drug is estimated to be unavailable for refill at some point during the prescription period. The alert may further comprise an alternative prescription drug that is not estimated to be unavailable, enabling proactive prescription modifications rather than reactive responses to drug shortages. Furthermore, the disclosed system integrates prescription rate information and manufacture rate information from various data sources, whether public or proprietary, to generate meaningful availability estimates for specific time periods, thereby overcoming the data integration limitations of existing systems.

In one aspect, a prescription drug availability estimation system is provided. The system includes at least one non-transitory computer readable medium and at least one database stored in the at least one non-transitory computer readable medium. The at least one database comprises prescription rate data information for a plurality of prescription drugs and manufacture rate data information for the plurality of prescription drugs. The system further includes at least one processor communicatively coupled with the at least one non-transitory computer readable medium. An estimation system is configured to be executed by the at least one processor to analyze prescription rate data for a first prescription drug and manufacture rate data for the first prescription drug to estimate an availability of the first prescription drug at each of a plurality of dates during a prescription period, and to identify one or more of the plurality of dates during the prescription period having a corresponding availability of the first prescription drug that is less than a first threshold. An alert system is configured to be executed by the at least one processor to provide an alert comprising a notice that the first prescription drug is estimated to be unavailable for refill at some point during the prescription period.

In another aspect, a computer implemented method for estimating an availability of prescription drugs is provided. The method includes receiving an indicator related to a prospective prescription of a first prescription drug, the prospective prescription having an amount of the first prescription drug and a prescription period including one or more refill dates. The method further includes obtaining prescription rate data for the first prescription drug from at least a first data source comprising prescription rate data information, and obtaining manufacture rate data for the first prescription drug from at least a second data source comprising manufacture rate data information. The method includes analyzing the prescription rate data and the manufacture rate data to estimate an availability of the first prescription drug at each of the one or more refill dates, and identifying one or more of the one or more refill dates having a corresponding availability of the first prescription drug that is less than a first threshold. The method further includes providing an alert in response to receiving the indicator, the alert comprising a notice that the first prescription drug is estimated to be unavailable for refill at some point during the prescription period.

In yet another aspect, a computer-implemented system is provided. The system includes means for identifying a first prescription drug and a prescription period, means for obtaining prescription rate data for the first prescription drug from at least a first data source comprising prescription rate data information, and means for obtaining manufacture rate data for the first prescription drug from at least a second data source comprising manufacture rate data information. The system further includes means for analyzing the prescription rate data and the manufacture rate data to estimate an availability of the first prescription drug at each of a plurality of dates during the prescription period, means for identifying one or more of the plurality of dates during the prescription period having a corresponding availability of the first prescription drug that is less than a first threshold, and means for providing an alert comprising a notice that the first prescription drug is estimated to be unavailable for refill at some point during the prescription period.

Other features and advantages of the present disclosure will become more readily apparent to those of ordinary skill in the art after reviewing the following detailed description and accompanying drawings.

### BRIEF DESCRIPTION OF THE DRAWINGS

The structure and operation of the present disclosure will be understood from a review of the following detailed description and the accompanying figures that include non-limiting and non-exhaustive examples.

FIG. 1 is an example network architecture of a system in which one or more of the processes described herein may be implemented, according to an embodiment.

FIG. 2 is an example processing system by which one or more of the processes described herein may be executed, according to an embodiment.

FIG. 3 is an example flowchart for a process for estimating prescription drug availability, according to an embodiment.

FIG. 4 is an example flowchart for a process for identifying alternative prescription drugs, according to an embodiment.

FIG. 5 is an example flowchart for a process for obtaining and integrating prescription rate data and manufacture rate data, according to an embodiment.

FIG. 6 is an example flowchart for a process for estimating regional prescription drug availability, according to an embodiment.

### DETAILED DESCRIPTION

A detailed description of systems, devices, methods, and computer readable media consistent with embodiments of the present disclosure is provided below. It should be recognized, however, that the detailed description is not intended as a limitation on the scope of the present disclosure and that the present description encompasses additional and alternative combinations and modifications to the embodiments and aspects expressly described herein.

While various embodiments are described, it should be understood that the present disclosure is not limited to any one embodiment, but instead encompasses numerous alternatives, modifications, and equivalents. In addition, while numerous specific details are set forth in the following description in order to provide a thorough understanding of the embodiments disclosed herein, some embodiments can be practiced without some or all of these details. Moreover, for the purpose of clarity, certain technical material that is known in the related art has not been described in detail in order to avoid unnecessarily obscuring the disclosure.

There are several challenges and technical problems that persist in the prior art. For one, conventional pharmaceutical supply chain management systems lack the ability to proactively analyze and compare prescription rate data with manufacture rate data to anticipate future drug availability issues. As a result, healthcare providers are often unaware of potential drug shortages until the shortage has already occurred, at which point patients may be unable to obtain their prescribed medications for refills during a prescription period. Furthermore, existing systems do not provide timely notifications to healthcare providers at the point of prescribing, which means physicians may prescribe drugs that will become unavailable before the prescription period ends, leading to disruptions in patient care and the reactive modification of prescriptions rather than proactive adjustments. Additionally, conventional systems do not adequately integrate prescription rate information with manufacture rate information from various data sources, whether public or proprietary, to generate meaningful availability estimates for specific time periods. The lack of data integration capabilities restricts the ability of healthcare providers, pharmacy benefit managers, and healthcare insurers to anticipate and respond to supply and demand imbalances before they affect patient care. These unresolved issues and technical problems underscore the demand for improved systems and methods for estimating prescription drug availability.

There are several technical advantages of the prescription drug availability estimation system of the present disclosure. First, the integration of prescription rate data analysis with manufacture rate data analysis enables proactive identification of potential drug shortages before they occur, allowing healthcare providers to anticipate availability issues rather than discovering them after a shortage has already materialized. This approach transforms pharmaceutical supply chain management from a reactive system to a predictive system, thereby reducing disruptions in patient care. Second, the alert system provides timely notifications to healthcare providers at the point of prescribing, which allows physicians to modify prescriptions in real time when a drug is estimated to be unavailable for refill at some point during the prescription period. The alert may further comprise an alternative prescription drug that is not estimated to be unavailable, enabling proactive prescription modifications rather than reactive responses to drug shortages. Third, the disclosed system accommodates data integration from various data sources, whether public or proprietary, by employing different access mechanisms such as direct access for public data sources and application programming interface (API) access for proprietary data sources. This flexibility in data acquisition allows the system to generate meaningful availability estimates for specific time periods by aggregating information from multiple sources. Fourth, the system may provide regional availability estimates by analyzing regional prescription rate data and regional manufacture rate data, which accounts for geographic variations in supply and demand conditions. Fifth, the alert system may provide notifications to various stakeholders including healthcare providers, pharmacy benefit managers, and healthcare insurers, thereby enabling coordinated responses to anticipated supply and demand imbalances. These technical advantages collectively contribute to an improved pharmaceutical supply chain management solution that enables proactive rather than reactive management of prescription drug availability.

The present disclosure provides a prescription drug availability estimation system that analyzes prescription rate data and manufacture rate data to estimate the availability of prescription drugs during a prescription period. The disclosed system improves device operation by enabling healthcare provider systems to receive real-time alerts at the point of prescribing when a prescription drug is estimated to be unavailable for refill at some point during the prescription period, thereby transforming pharmaceutical supply chain management from a reactive system to a predictive system. The alert system may further provide the name of a suitable alternative prescription drug that is not estimated to be unavailable, allowing physicians to modify prescriptions in real time rather than discovering availability issues after a shortage has already materialized. The disclosed system may reduce the number of CPU cycles for computational aspects by integrating prescription rate data information and manufacture rate data information from various data sources into at least one database, which allows the estimation system to perform availability analysis using locally stored data rather than repeatedly querying multiple external systems for each availability estimation. Additionally, the system accommodates data integration from both public data sources and proprietary data sources by employing different access mechanisms, such as direct access for public data sources and application programming interface (API) access for proprietary data sources, which provides flexibility in data acquisition while maintaining computational efficiency. The system may also provide regional availability estimates by analyzing regional prescription rate data and regional manufacture rate data, which accounts for geographic variations in supply and demand conditions. Furthermore, the alert system may provide notifications to various stakeholders including healthcare providers, pharmacy benefit managers, and healthcare insurers, thereby enabling coordinated responses to anticipated supply and demand imbalances. These improvements are achieved through the combination of the estimation system that analyzes prescription rate data and manufacture rate data to estimate availability at each of a plurality of dates during a prescription period, and the alert system that provides timely notifications when the estimated availability falls below a threshold, which together form the core components of the disclosed prescription drug availability estimation system.

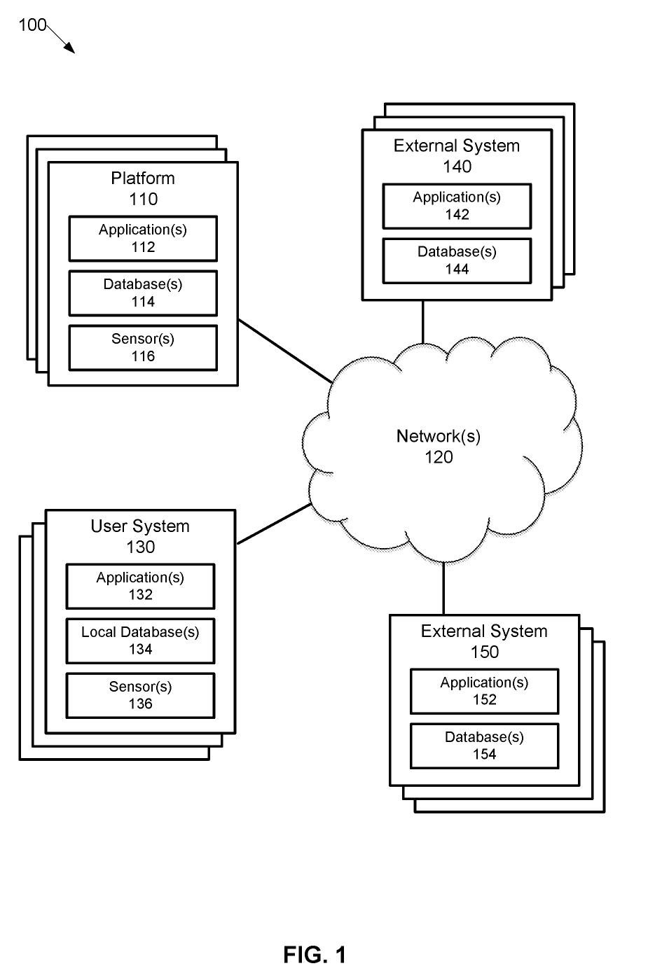

FIG. 1 illustrates an example infrastructure 100 in which one or more of the disclosed processes may be implemented, according to various embodiments. The infrastructure 100 comprises a platform 110, network(s) 120, a user system 130, an external system 140, and an external system 150, all communicatively connected via the network(s) 120.

The platform 110 serves as the main server of the prescription drug availability estimation system, orchestrating data processing and system operations for estimating prescription drug availability. The platform 110 comprises application(s) 112, database(s) 114, and sensor(s) 116. The platform 110 may comprise dedicated hardware and software, or may instead comprise cloud instances which utilize shared resources of one or more servers. The platform 110 devices, servers, and/or cloud instances may be collocated and/or geographically distributed. The application(s) 112 hosted on the platform 110 may execute one or more of the various functions, processes, methods, and/or software modules described herein, including the estimation system that analyzes prescription rate data and manufacture rate data to estimate the availability of prescription drugs during a prescription period, and the alert system that provides notifications when estimated availability falls below a threshold. The database(s) 114 may store prescription rate data information and manufacture rate data information for a plurality of prescription drugs. In some aspects, the database(s) 114 integrates prescription rate data information and manufacture rate data information from various data sources, which allows the estimation system to perform availability analysis using locally stored data rather than repeatedly querying multiple external systems for each availability estimation. The sensor(s) 116 may be communicatively coupled to the platform 110 to provide data inputs.

The user system 130 functions as the primary user interface of the prescription drug availability estimation system, enabling healthcare providers and other users to interact with the system. The user system 130 comprises application(s) 132, local database(s) 134, and sensor(s) 136. The user system 130 may comprise any type of computing device capable of wired and/or wireless communication, including desktop computers, laptop computers, tablet computers, smart phones or other mobile devices, servers, and the like. The application(s) 132 executing on the user system 130 may interact with the application(s) 112 executing on the platform 110 to receive real-time alerts at the point of prescribing when a prescription drug is estimated to be unavailable for refill at some point during the prescription period. The local database(s) 134 provides local data storage on the user system 130. The sensor(s) 136 may include devices such as cameras or other input devices.

The external system 140 and the external system 150 encompass external data repositories and external resources that provide prescription rate data information and manufacture rate data information to the platform 110. The external system 140 comprises application(s) 142 and database(s) 144. The external system 150 comprises application(s) 152 and database(s) 154. The external systems 140 and 150 may comprise servers which host one or more websites and/or web services. In one aspect, the external system 140 stores prescription rate data information corresponding to one or more prescription drugs and the external system 150 stores manufacture rate data information corresponding to one or more prescription drugs. The external systems 140 and 150 may be public data sources or may be private data sources storing proprietary rate data information. The platform 110 may obtain information from the external systems 140 and 150 via an application programming interface (API) or other software implemented means. In some aspects, for public data sources, the platform 110 obtains prescription rate data and manufacture rate data via direct access, while for proprietary data sources, the platform 110 obtains the data via API access. This flexibility in data acquisition allows the system to generate meaningful availability estimates for specific time periods by aggregating information from multiple sources.

The communication flow between the components of the infrastructure 100 operates as follows. The user system 130 transmits user requests to the platform 110 via the network(s) 120, such as requests related to a prospective prescription of a prescription drug. The platform 110 processes these requests and communicates with the external systems 140 and 150 via the network(s) 120 to retrieve prescription rate data information and manufacture rate data information. The platform 110 then consolidates the processed information, performs availability analysis using the estimation system, and transmits responses back to the user system 130, including alerts when a prescription drug is estimated to be unavailable for refill at some point during the prescription period. The alert may further comprise an alternative prescription drug that is not estimated to be unavailable, allowing healthcare providers to modify prescriptions in real time rather than discovering availability issues after a shortage has already materialized.

The network(s) 120 may comprise a personal area network, a mesh network, a local area network, a wide area network, the Internet, and any combination of wired or wireless networks. The platform 110 may communicate with the user system 130 and the external systems 140 and 150 through the network(s) 120 using standard transmission protocols.

The technical solutions provided by the infrastructure 100 address the problem of fragmented data access by providing a unified interface that integrates prescription rate data information and manufacture rate data information from multiple external resources. This approach eliminates the need for healthcare providers to manage separate connections to various data systems, thereby reducing latency and improving system reliability through centralized error handling and data validation. Additionally, by integrating prescription rate data information and manufacture rate data information from various data sources into the database(s) 114, the platform 110 may reduce the number of CPU cycles for computational aspects by allowing the estimation system to perform availability analysis using locally stored data rather than repeatedly querying multiple external systems for each availability estimation. The alert system may provide notifications to various stakeholders including healthcare providers, pharmacy benefit managers, and healthcare insurers, thereby enabling coordinated responses to anticipated supply and demand imbalances. Furthermore, the system may provide regional availability estimates by analyzing regional prescription rate data and regional manufacture rate data, which accounts for geographic variations in supply and demand conditions.

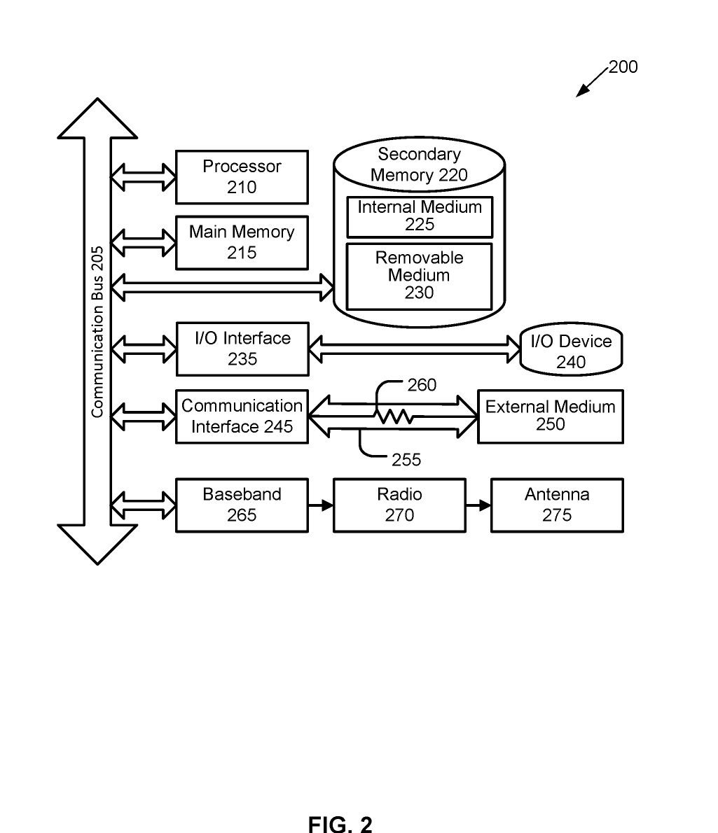

FIG. 2 is a block diagram illustrating an example wired or wireless system 200 that may be used in connection with various embodiments described herein. The system 200 may be used as or in conjunction with one or more of the functions, processes, or methods described herein, such as to store and/or execute the estimation system, the alert system, or one or more software modules of the prescription drug availability estimation system. The system 200 may represent components of the platform 110, the user system 130, the external system 140, the external system 150, and/or other processing devices described herein. The system 200 can be any type of processor-enabled device that is capable of executing instructions and capable of wired or wireless data communication. For example, the system 200 can be an application specific integrated circuit (ASIC), a custom built computer, a conventional personal computer, a conventional server computer, or the like. Other processor enabled computer systems and/or architectures may also be used for the system 200, as will be clear to those skilled in the art.

The system 200 includes one or more processors, such as processor 210. Additional processors may be provided, such as an auxiliary processor to manage input/output, an auxiliary processor to perform floating-point mathematical operations, a special-purpose microprocessor having an architecture suitable for fast execution of signal-processing algorithms (e.g., digital-signal processor), a slave processor subordinate to the main processing system (e.g., back-end processor), an additional microprocessor or controller for dual or multiple processor systems, and/or a coprocessor. Such auxiliary processors may be discrete processors or may be integrated with the processor 210.

The processor 210 is connected to a communication bus 205. The communication bus 205 may include a data channel for facilitating information transfer between storage and other peripheral components of the system 200. Furthermore, the communication bus 205 may provide a set of signals used for communication with the processor 210, including a data bus, address bus, and/or control bus. The communication bus 205 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, standards promulgated by the Institute of Electrical and Electronics Engineers (IEEE) including IEEE 488 general-purpose interface bus (GPIB), IEEE 696/S-100, and/or the like.

The system 200 includes a main memory 215 and may also include a secondary memory 220. The main memory 215 provides storage of instructions and data for programs executing on the processor 210, such as one or more of the functions and/or modules discussed herein, including the estimation system that analyzes prescription rate data and manufacture rate data to estimate the availability of prescription drugs during a prescription period, and the alert system that provides notifications when estimated availability falls below a threshold. It should be understood that programs stored in the memory and executed by the processor 210 may be written and/or compiled according to any suitable language, including without limitation C/C++, Java, JavaScript, Perl, Visual Basic, .NET, and the like. The main memory 215 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

The secondary memory 220 may optionally include an internal medium 225 and/or a removable medium 230. The removable medium 230 is read from and/or written to in any well-known manner. The removable storage medium 230 may be, for example, a magnetic tape drive, a compact disc (CD) drive, a digital versatile disc (DVD) drive, other optical drive, a flash memory drive, and/or the like.

The secondary memory 220 is a non-transitory computer-readable medium having computer-executable code (e.g., disclosed software modules including the estimation system and the alert system) and/or other data stored thereon. The computer software or data stored on the secondary memory 220 is read into the main memory 215 for execution by the processor 210.

In alternative embodiments, the secondary memory 220 may include other similar means for allowing computer programs or other data or instructions to be loaded into the system 200. Such means may include, for example, a communication interface 245, which allows software and data to be transferred from an external storage medium 250 to the system 200. Examples of the external storage medium 250 may include an external hard disk drive, an external optical drive, an external magneto-optical drive, and/or the like. Other examples of the secondary memory 220 may include semiconductor-based memory, such as programmable read-only memory (PROM), erasable programmable read-only memory (EPROM), electrically erasable read-only memory (EEPROM), and flash memory (block-oriented memory similar to EEPROM).

As mentioned above, the system 200 may include a communication interface 245. The communication interface 245 allows software and data to be transferred between the system 200 and external devices (e.g., printers), networks, or other information sources. For example, computer software or executable code may be transferred to the system 200 from a network server (e.g., the platform 110) via the communication interface 245. Examples of the communication interface 245 include a built-in network adapter, network interface card (NIC), Personal Computer Memory Card International Association (PCMCIA) network card, card bus network adapter, wireless network adapter, Universal Serial Bus (USB) network adapter, modem, a wireless data card, a communications port, an infrared interface, an IEEE 1394 fire-wire, and any other device capable of interfacing the system 200 with a network (e.g., the network(s) 120) or another computing device. The communication interface 245 may implement industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software and data transferred via the communication interface 245 are generally in the form of electrical communication signals 260. These signals 260 may be provided to the communication interface 245 via a communication channel 255. In an embodiment, the communication channel 255 may be a wired or wireless network (e.g., the network(s) 120), or any variety of other communication links. The communication channel 255 carries signals 260 and can be implemented using a variety of wired or wireless communication means including wire or cable, fiber optics, conventional phone line, cellular phone link, wireless data communication link, radio frequency (RF) link, or infrared link, just to name a few.

Computer-executable code (e.g., computer programs, such as the disclosed estimation system and alert system, or software modules) is stored in the main memory 215 and/or the secondary memory 220. Computer programs can also be received via the communication interface 245 and stored in the main memory 215 and/or the secondary memory 220. Such computer programs, when executed, enable the system 200 to perform the various functions of the disclosed embodiments as described elsewhere herein, including analyzing prescription rate data and manufacture rate data to estimate the availability of prescription drugs during a prescription period, identifying dates having a corresponding availability less than a threshold, and providing alerts comprising a notice that a prescription drug is estimated to be unavailable for refill at some point during the prescription period.

In this description, the term "computer-readable medium" is used to refer to any non-transitory computer-readable storage media used to provide computer-executable code and/or other data to or within the system 200. Examples of such media include the main memory 215, the secondary memory 220 (including the internal medium 225, the removable medium 230, and the external storage medium 250), and any peripheral device communicatively coupled with the communication interface 245 (including a network information server or other network device). These non-transitory computer-readable media are means for providing executable code, programming instructions, software, and/or other data to the system 200.

In an embodiment that is implemented using software, the software may be stored on a computer-readable medium and loaded into the system 200 by way of the removable medium 230, an I/O interface 235, or the communication interface 245. In such an embodiment, the software is loaded into the system 200 in the form of electrical communication signals 260. The software, when executed by the processor 210, causes the processor 210 to perform one or more of the processes and functions described elsewhere herein.

In an embodiment, the I/O interface 235 provides an interface between one or more components of the system 200 and one or more input and/or output devices 240. Example input devices include, without limitation, sensors, keyboards, touch screens or other touch-sensitive devices, biometric sensing devices, computer mice, trackballs, pen-based pointing devices, and/or the like. Examples of output devices include, without limitation, other processing devices, cathode ray tubes (CRTs), plasma displays, light-emitting diode (LED) displays, liquid crystal displays (LCDs), printers, vacuum fluorescent displays (VFDs), surface-conduction electron-emitter displays (SEDs), field emission displays (FEDs), head mounted displays (HMDs), and/or the like. In some cases, an input and output device 240 may be combined, such as in the case of a touch panel display (e.g., in a smartphone, tablet, or other mobile device).

In an embodiment, the I/O device 240 may be any type of external or integrated display and may include one or more discrete displays that in aggregate form the I/O device 240. The I/O device 240 may be capable of 2D or 3D presentation of visual information to a user of the system 200. In one embodiment, the I/O device 240 may be a virtual reality or augmented reality device in the form of an HMD worn by the user so the user may visualize the presentation of information in 3D.

The system 200 may also include optional wireless communication components that facilitate wireless communication over a voice network and/or a data network (e.g., in the case of the user system 130). The wireless communication components comprise an antenna system 275, a radio system 270, and a baseband system 265. In the system 200, radio frequency (RF) signals are transmitted and received over the air by the antenna system 275 under the management of the radio system 270.

In an embodiment, the antenna system 275 may comprise one or more antennae and one or more multiplexors that perform a switching function to provide the antenna system 275 with transmit and receive signal paths. In the receive path, received RF signals can be coupled from a multiplexor to a low noise amplifier that amplifies the received RF signal and sends the amplified signal to the radio system 270.

In an alternative embodiment, the radio system 270 may comprise one or more radios that are configured to communicate over various frequencies. In an embodiment, the radio system 270 may combine a demodulator and modulator in one integrated circuit (IC). The demodulator and modulator can also be separate components. In the incoming path, the demodulator strips away the RF carrier signal leaving a baseband receive audio signal, which is sent from the radio system 270 to the baseband system 265.

If the received signal contains audio information, then the baseband system 265 decodes the signal and converts it to an analog signal. Then the signal is amplified and sent to a speaker. The baseband system 265 also receives analog audio signals from a microphone. These analog audio signals are converted to digital signals and encoded by the baseband system 265. The baseband system 265 also encodes the digital signals for transmission and generates a baseband transmit audio signal that is routed to the modulator portion of the radio system 270. The modulator mixes the baseband transmit audio signal with an RF carrier signal, generating an RF transmit signal that is routed to the antenna system 275 and may pass through a power amplifier. The power amplifier amplifies the RF transmit signal and routes it to the antenna system 275, where the signal is switched to the antenna port for transmission.

The baseband system 265 is also communicatively coupled with the processor 210, which may be a central processing unit (CPU). The processor 210 has access to data storage areas 215 and 220. The processor 210 is configured to execute instructions (i.e., computer programs, such as the disclosed estimation system and alert system, or software modules) that can be stored in the main memory 215 or the secondary memory 220. Computer programs can also be received from the baseband system 265 and stored in the main memory 215 or in the secondary memory 220, or executed upon receipt. Such computer programs, when executed, enable the system 200 to perform the various functions of the disclosed embodiments.

The system 200, when implemented as the platform 110, the user system 130, the external system 140, or the external system 150, provides several operational improvements for the prescription drug availability estimation system. By integrating prescription rate data information and manufacture rate data information from various data sources into the main memory 215 or the secondary memory 220, the system 200 may reduce the number of CPU cycles for computational aspects by allowing the estimation system to perform availability analysis using locally stored data rather than repeatedly querying multiple external systems for each availability estimation. The communication interface 245 enables the system 200 to receive prescription rate data and manufacture rate data from both public data sources and proprietary data sources, with the flexibility to employ different access mechanisms such as direct access for public data sources and application programming interface (API) access for proprietary data sources. When the system 200 is implemented as the user system 130, the communication interface 245 enables healthcare providers to receive real-time alerts at the point of prescribing when a prescription drug is estimated to be unavailable for refill at some point during the prescription period, thereby transforming pharmaceutical supply chain management from a reactive system to a predictive system. The alert may further comprise an alternative prescription drug that is not estimated to be unavailable, allowing physicians to modify prescriptions in real time rather than discovering availability issues after a shortage has already materialized.

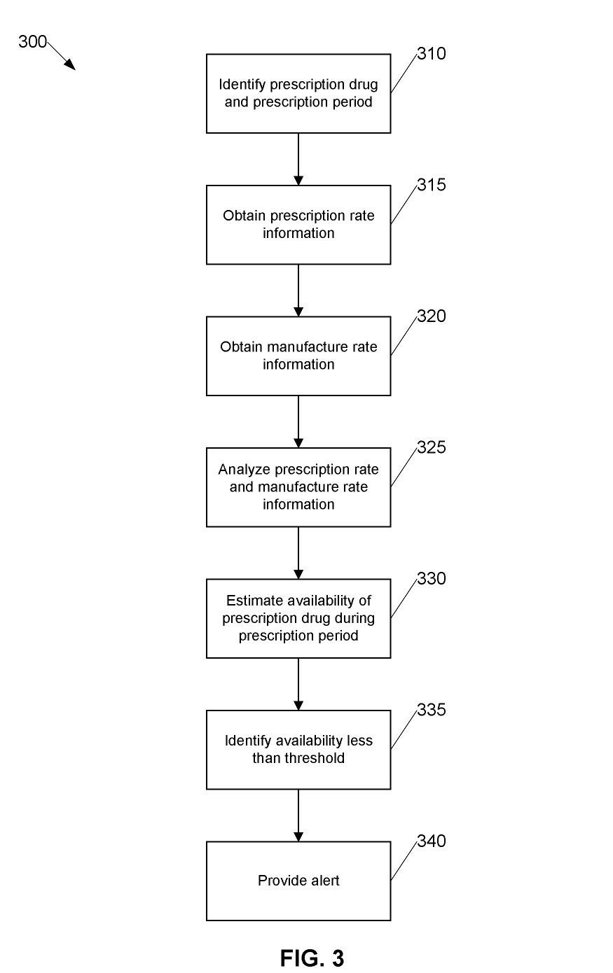

FIG. 3 is a flow diagram illustrating an example process 300 for estimating prescription drug availability according to an embodiment. In one aspect, the process of FIG. 3 may be carried out by the system described with respect to FIG. 1 in combination with one or more processing devices described with respect to FIG. 2 that may be programmed to execute special purpose software instructions.

Initially, at 310 the system identifies a prescription drug and a prescription period. The prescription drug may be a drug that is being proposed to be prescribed in real time or a drug that is being proactively analyzed to estimate its future availability. The prescription period may be a number of days or some other time period.

At 315, the system obtains prescription rate information for the prescription drug. The prescription rate information may be obtained from a local data storage area or from one or more external systems, which may be public or private databases. This data acquisition approach addresses the technical problem of fragmented data access by enabling the system to retrieve information from multiple sources through a unified interface.

At 320, the system obtains manufacture rate information for the prescription drug. Similar to the prescription rate information, the manufacture rate information may be obtained from a local data storage area or from one or more external systems. By integrating both prescription rate information and manufacture rate information, the system enables comprehensive supply and demand analysis that conventional systems lack.

At 325, the system analyzes the prescription rate information and the manufacture rate information. This analysis compares the rate at which the prescription drug is being prescribed against the rate at which it is being manufactured. The analysis transforms pharmaceutical supply chain management from a reactive system to a predictive system by enabling proactive identification of potential drug shortages before they occur.

At 330, the system estimates the availability of the prescription drug during the prescription period based on the analysis performed at 325. The estimation determines whether there will be a surplus or deficit of the prescription drug at various times during the prescription period, such as at anticipated refill date ranges.

At 335, the system identifies whether the availability of the prescription drug is less than a threshold at some point during the prescription period. The system determines if the severity of any deficit exceeds a predetermined risk threshold, thereby enabling healthcare providers to anticipate availability issues rather than discovering them after a shortage has already materialized.

At 340, the system provides an alert in response to identifying that the availability falls below the threshold. The alert notifies the user that the prescription drug will likely not be available for a refill during the prescription period and may additionally provide the name of a suitable alternative prescription drug that will be available during the prescription period. This alert mechanism addresses the technical problem of conventional systems that fail to provide timely notifications to healthcare providers at the point of prescribing.

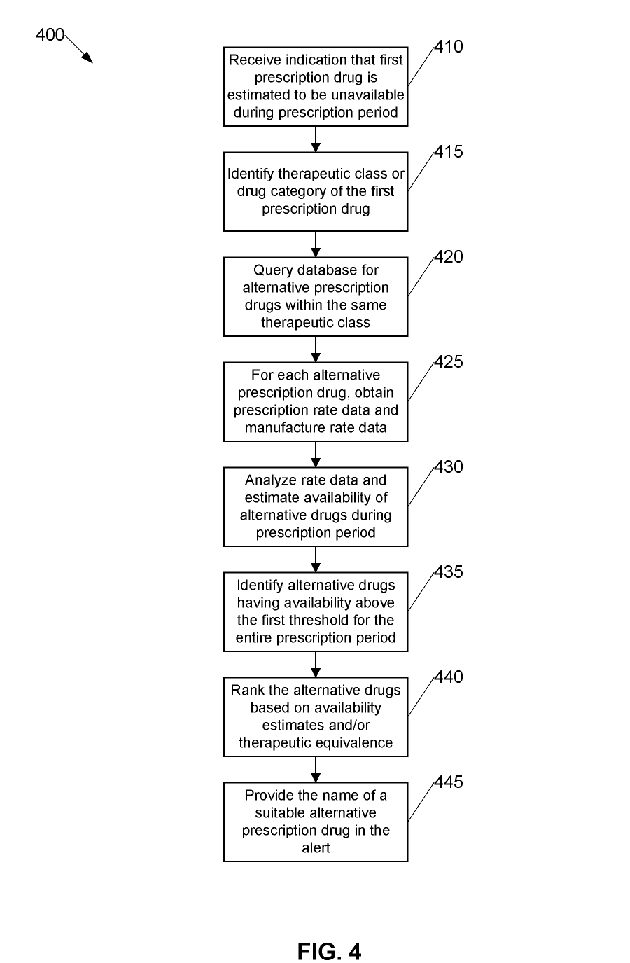

FIG. 4 is a flow diagram illustrating an example process 400 for identifying alternative prescription drugs when a first prescription drug is estimated to be unavailable according to an embodiment. In one aspect, the process of FIG. 4 may be carried out by the system described with respect to FIG. 1 in combination with one or more processing devices described with respect to FIG. 2 that may be programmed to execute special purpose software instructions.

Initially, at 410 the system receives an indication that a first prescription drug is estimated to be unavailable during a prescription period. This indication may be generated by the estimation system described with respect to FIG. 3.

At 415, the system identifies a therapeutic class or drug category of the first prescription drug. The therapeutic class provides a basis for identifying drugs that may serve as suitable alternatives to the first prescription drug.

At 420, the system queries a database for alternative prescription drugs within the same therapeutic class. The database may be the database(s) 114 stored on the platform 110 or may be accessed from one or more external systems.

At 425, for each alternative prescription drug, the system obtains prescription rate data and manufacture rate data. This data acquisition enables the system to perform availability analysis for each potential alternative drug.

At 430, the system analyzes the rate data and estimates availability of the alternative drugs during the prescription period. This analysis applies the same estimation techniques used for the first prescription drug to each alternative drug.

At 435, the system identifies alternative drugs having availability above the first threshold for the prescription period. This identification filters the alternative drugs to those that are estimated to remain available throughout the prescription period.

At 440, the system ranks the alternative drugs based on availability estimates and/or therapeutic equivalence. The ranking provides healthcare providers with prioritized alternatives that are both therapeutically suitable and estimated to be available.

At 445, the system provides the name of a suitable alternative prescription drug in the alert. The process 400 enables the system to suggest therapeutically equivalent alternatives that are estimated to remain available throughout the prescription period, thereby allowing healthcare providers to proactively modify prescriptions when the originally prescribed drug is estimated to be unavailable.

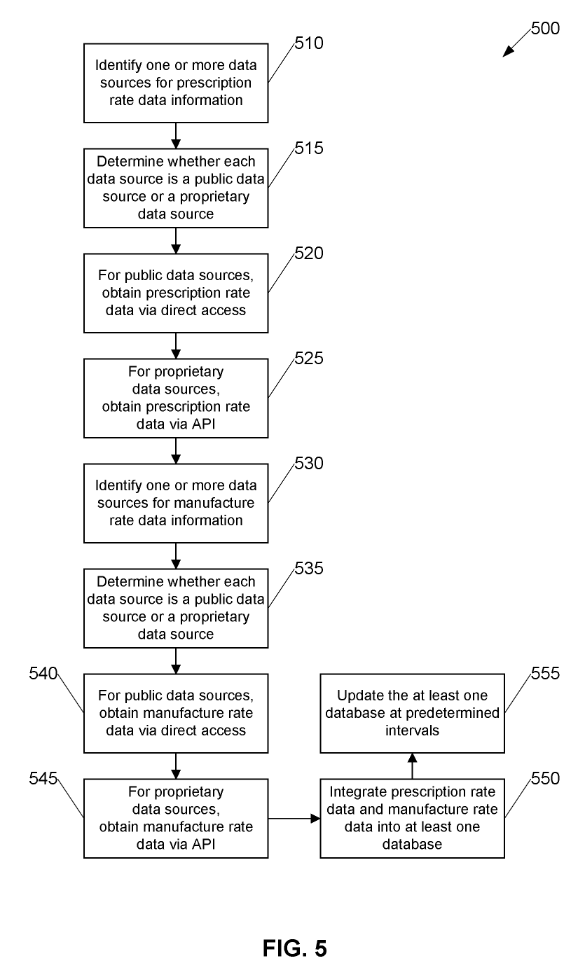

FIG. 5 is a flow diagram illustrating an example process 500 for obtaining and integrating prescription rate data and manufacture rate data from various data sources according to an embodiment. In one aspect, the process of FIG. 5 may be carried out by the system described with respect to FIG. 1 in combination with one or more processing devices described with respect to FIG. 2 that may be programmed to execute special purpose software instructions.

Initially, at 510 the system identifies one or more data sources for prescription rate data information. The data sources may include public databases, proprietary databases, or a combination thereof.

At 515, the system determines whether each data source is a public data source or a proprietary data source. This determination enables the system to employ the appropriate access mechanism for each data source.

At 520, for public data sources, the system obtains prescription rate data via direct access. Direct access may involve querying publicly available databases or retrieving data from open data repositories.

At 525, for proprietary data sources, the system obtains prescription rate data via API. The API access mechanism enables the system to retrieve data from proprietary sources while respecting access controls and data usage agreements.

At 530, the system identifies one or more data sources for manufacture rate data information. Similar to the prescription rate data sources, the manufacture rate data sources may include public databases, proprietary databases, or a combination thereof.

At 535, the system determines whether each data source is a public data source or a proprietary data source. This determination enables the system to employ the appropriate access mechanism for each manufacture rate data source.

At 540, for public data sources, the system obtains manufacture rate data via direct access. The direct access mechanism provides efficient retrieval of publicly available manufacture rate information.

At 545, for proprietary data sources, the system obtains manufacture rate data via API. The API access mechanism enables secure retrieval of proprietary manufacture rate information.

At 550, the system integrates prescription rate data and manufacture rate data into at least one database. The integration consolidates data from multiple sources into a unified data store, which addresses the technical problem of fragmented data access in conventional systems. By integrating the data into at least one database, the system may reduce the number of CPU cycles for computational aspects by allowing the estimation system to perform availability analysis using locally stored data rather than repeatedly querying multiple external systems for each availability estimation.

At 555, the system updates the at least one database at predetermined intervals. The periodic updating ensures that the prescription rate data and manufacture rate data remain current for accurate availability estimation. The process 500 accommodates both public and proprietary data sources by employing different access mechanisms, with direct access used for public data sources and API access used for proprietary data sources.

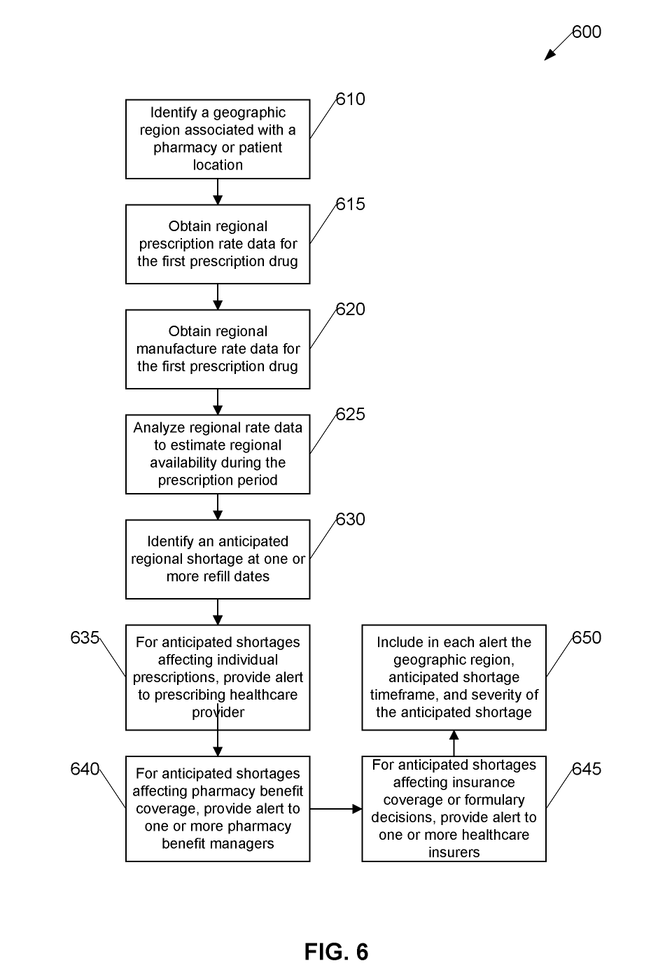

FIG. 6 is a flow diagram illustrating an example process 600 for estimating regional prescription drug availability according to an embodiment. In one aspect, the process of FIG. 6 may be carried out by the system described with respect to FIG. 1 in combination with one or more processing devices described with respect to FIG. 2 that may be programmed to execute special purpose software instructions.

Initially, at 610 the system identifies a geographic region associated with a pharmacy or patient location. This establishes the regional context for the availability analysis, which accounts for geographic variations in supply and demand conditions.

At 615, the system obtains regional prescription rate data for the first prescription drug. The regional prescription rate data provides information about how frequently the prescription drug is being prescribed within the identified geographic region.

At 620, the system obtains regional manufacture rate data for the first prescription drug. This data reflects the manufacturing output and distribution of the prescription drug within the relevant geographic region.

At 625, the system analyzes the regional rate data to estimate regional availability during the prescription period. This analysis compares the regional prescription rate data with the regional manufacture rate data to determine anticipated supply and demand conditions within the geographic region.

At 630, the system identifies an anticipated regional shortage at one or more refill dates based on the analysis performed at 625. This identification determines specific time periods when the prescription drug may not be available for refill within the geographic region.

At 635, for anticipated shortages affecting individual prescriptions, the system provides an alert to the prescribing healthcare provider. This alert enables the healthcare provider to modify the prescription in real time rather than discovering availability issues after a shortage has already materialized.

At 640, for anticipated shortages affecting pharmacy benefit coverage, the system provides an alert to one or more pharmacy benefit managers. This notification enables pharmacy benefit managers to anticipate and respond to supply and demand imbalances before they affect patient care.

At 645, for anticipated shortages affecting insurance coverage or formulary decisions, the system provides an alert to one or more healthcare insurers. This notification enables healthcare insurers to coordinate responses to anticipated supply and demand imbalances.

At 650, the system includes in each alert the geographic region, anticipated shortage timeframe, and severity of the anticipated shortage. This information enables the recipients of the alerts to take appropriate action based on the regional availability estimates. The process 600 provides regional availability estimates that account for geographic variations in supply and demand conditions, thereby enabling coordinated responses to anticipated regional shortages among healthcare providers, pharmacy benefit managers, and healthcare insurers.

The above description of the disclosed embodiments is provided to enable a person skilled in the art to make or use the invention. Various modifications to the disclosed embodiments will be readily apparent to those skilled in the art, and the principles described herein can be applied to alternative embodiments and/or modified embodiments without departing from the spirit or scope of the invention. Thus, it is to be understood that the description and drawings presented herein represent an embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

What is claimed is:

1. A computer implemented method for estimating an availability of prescription drugs comprising:
receiving an indicator related to a prospective prescription of a first prescription drug, the prospective prescription having an amount of the first prescription drug and a prescription period including one or more refill dates;
obtaining prescription rate data for the first prescription drug from at least a first data source comprising prescription rate data information;
obtaining manufacture rate data for the first prescription drug from at least a second data source comprising manufacture rate data information;
analyzing the prescription rate data and the manufacture rate data to estimate an availability of the first prescription drug at each of the one or more refill dates;
identifying one or more of the one or more refill dates having a corresponding availability of the first prescription drug that is less than a first threshold; and
providing an alert in response to receiving the indicator, the alert comprising a notice that the first prescription drug is estimated to be unavailable for refill at some point during the prescription period.

2. The method of claim 1, wherein each of the one or more refill dates comprises a plurality of days.

3. The method of claim 1, wherein one or more of the first data source and the second data source are public data sources.

4. The method of claim 1, wherein one or more of the first data source and the second data source are proprietary data sources.

5. The method of claim 1, wherein the alert further comprises an alternative to the first prescription drug that is not estimated to be unavailable for refill at some point during the prescription period.

6. A prescription drug availability estimation system comprising:
at least one non-transitory computer readable medium;
at least one database stored in the at least one non-transitory computer readable medium, the at least one database comprising prescription rate data information for a plurality of prescription drugs and manufacture rate data information for the plurality of prescription drugs;
at least one processor communicatively coupled with the at least one non-transitory computer readable medium;
an estimation system configured to be executed by the at least one processor to analyze the prescription rate data for a first prescription drug and the manufacture rate data for the first prescription drug to estimate an availability of the first prescription drug at each of a plurality of dates during a prescription period and identify one or more of the plurality of dates during the prescription period having a corresponding availability of the first prescription drug that is less than a first threshold; and
an alert system configured to be executed by the at least one processor to provide an alert comprising a notice that the first prescription drug is estimated to be unavailable for refill at some point during the prescription period.

7. The system of claim 6, wherein the prescription period comprises at least three months.

8. The system of claim 6, wherein one or more of the prescription rate data information and the manufacture rate data information are obtained from public data sources and stored in the database.

9. The system of claim 6, wherein one or more of the prescription rate data information and the manufacture rate data information are obtained from proprietary data sources and stored in the database.

10. The system of claim 6, wherein the alert further comprises an alternative to the first prescription drug that is not estimated to be unavailable for refill at some point during the prescription period.

11. A system comprising at least one processor communicatively coupled with at least one non-transitory computer readable medium, wherein the processor is programmed to perform steps comprising:
receiving an indicator related to a prospective prescription of a first prescription drug, the prospective prescription having an amount of the first prescription drug and a prescription period including one or more refill dates;
obtaining prescription rate data for the first prescription drug from at least a first data source comprising prescription rate data information;
obtaining manufacture rate data for the first prescription drug from at least a second data source comprising manufacture rate data information;
analyzing the prescription rate data and the manufacture rate data to estimate an availability of the first prescription drug at each of the one or more refill dates;
identifying one or more of the one or more refill dates having a corresponding availability of the first prescription drug that is less than a first threshold; and
providing an alert in response to receiving the indicator, the alert comprising a notice that the first prescription drug is estimated to be unavailable for refill at some point during the prescription period.

12. A non-transitory computer readable medium having stored thereon one or more sequences of instructions for causing one or more processors to perform steps comprising:
receiving an indicator related to a prospective prescription of a first prescription drug, the prospective prescription having an amount of the first prescription drug and a prescription period including one or more refill dates;
obtaining prescription rate data for the first prescription drug from at least a first data source comprising prescription rate data information;
obtaining manufacture rate data for the first prescription drug from at least a second data source comprising manufacture rate data information;
analyzing the prescription rate data and the manufacture rate data to estimate an availability of the first prescription drug at each of the one or more refill dates;
identifying one or more of the one or more refill dates having a corresponding availability of the first prescription drug that is less than a first threshold; and
providing an alert in response to receiving the indicator, the alert comprising a notice that the first prescription drug is estimated to be unavailable for refill at some point during the prescription period.

13. A computer implemented method for estimating an availability of prescription drugs comprising:
identifying a first prescription drug;
identifying a prescription period;
obtaining prescription rate data for the first prescription drug from at least a first data source comprising prescription rate data information;
obtaining manufacture rate data for the first prescription drug from at least a second data source comprising manufacture rate data information;
analyzing the prescription rate data and the manufacture rate data to estimate an availability of the first prescription drug at each of a plurality of dates during the prescription period;
identifying one or more of the plurality of dates during the prescription period having a corresponding availability of the first prescription drug that is less than a first threshold; and
providing an alert comprising a notice that the first prescription drug is estimated to be unavailable for refill at some point during the prescription period.

14. The method of claim 13, wherein the prescription period comprises at least three months.

15. The method of claim 13, wherein the alert is provided to one or more pharmacy benefit managers.

16. The method of claim 13, wherein the alert is provided to one or more healthcare insurers.

17. The method of claim 13, wherein the alert further comprises an alternative to the first prescription drug that is not estimated to be unavailable for refill at some point during the prescription period.

18. A prescription drug availability estimation system comprising:
at least one non-transitory computer readable medium;
at least one database stored in the at least one non-transitory computer readable medium, the at least one database comprising prescription rate data information for a plurality of prescription drugs and manufacture rate data information for the plurality of prescription drugs;
at least one processor communicatively coupled with the at least one non-transitory computer readable medium;
an estimation system configured to be executed by the at least one processor to analyze the prescription rate data for a first prescription drug and the manufacture rate data for the first prescription drug to estimate an availability of the first prescription drug at each of a plurality of dates during a prescription period and identify one or more of the plurality of dates during the prescription period having a corresponding availability of the first prescription drug that is less than a first threshold; and
an alert system configured to be executed by the at least one processor to provide an alert comprising a notice that the first prescription drug is estimated to be unavailable for refill at some point during the prescription period.

19. The system of claim 18, wherein the prescription period comprises at least three months.

20. The system of claim 18, wherein one or more of the prescription rate data information and the manufacture rate data information are obtained from public data sources and stored in the database.

21. The system of claim 18, wherein one or more of the prescription rate data information and the manufacture rate data information are obtained from proprietary data sources and stored in the database.

22. The system of claim 18, wherein the alert further comprises an alternative to the first prescription drug that is not estimated to be unavailable for refill at some point during the prescription period.

23. A system comprising at least one processor communicatively coupled with at least one non-transitory computer readable medium, wherein the processor is programmed to perform steps comprising:
identifying a first prescription drug;
identifying a prescription period;
obtaining prescription rate data for the first prescription drug from at least a first data source comprising prescription rate data information;
obtaining manufacture rate data for the first prescription drug from at least a second data source comprising manufacture rate data information;
analyzing the prescription rate data and the manufacture rate data to estimate an availability of the first prescription drug at each of a plurality of dates during the prescription period;
identifying one or more of the plurality of dates during the prescription period having a corresponding availability of the first prescription drug that is less than a first threshold; and
providing an alert comprising a notice that the first prescription drug is estimated to be unavailable for refill at some point during the prescription period.

24. A non-transitory computer readable medium having stored thereon one or more sequences of instructions for causing one or more processors to perform steps comprising:
identifying a first prescription drug;
identifying a prescription period;
obtaining prescription rate data for the first prescription drug from at least a first data source comprising prescription rate data information;
obtaining manufacture rate data for the first prescription drug from at least a second data source comprising manufacture rate data information;
analyzing the prescription rate data and the manufacture rate data to estimate an availability of the first prescription drug at each of a plurality of dates during the prescription period;
identifying one or more of the plurality of dates during the prescription period having a corresponding availability of the first prescription drug that is less than a first threshold; and
providing an alert comprising a notice that the first prescription drug is estimated to be unavailable for refill at some point during the prescription period.
